(19) United States
(12) Patent Application Publication
(10) Pub. No.: US 2023/0143800 A1
(43) Pub. Date: May 11, 2023

(54) SYSTEMS, MEDIA, METHODS, AND APPARATUS FOR ENHANCED DIGITAL MICROSCOPY

(71) Applicant: Mikroscan Technologies, Inc., Vista, CA (US)

(72) Inventor: Victor CASAS, Vista, CA (US)

(21) Appl. No.: 18/148,297

(22) Filed: Dec. 29, 2022

Related U.S. Application Data
(63) Continuation of application No. 15/294,541, filed on Oct. 14, 2016, now abandoned.
(60) Provisional application No. 62/242,968, filed on Oct. 16, 2015.

(51) Int. Cl. G02B 21/24; G16H 80/00; G06V 10/25; G06V 20/69; H04L 65/61; H04L 65/613; G06T 7/90; G02B 21/08; G02B 21/36

(52) U.S. Cl. CPC G02B 21/241; G16H 80/00; G06V 10/25; G06V 20/69; G06V 20/698; H04L 65/61; H04L 65/613; G06T 7/90; G02B 21/086; G02B 21/367; G02B 21/368; G06T 11/60

(57) ABSTRACT

Described herein are improvements in digital microscopy and telepathology. The disclosed technologies enable users to configure digital microscopes remotely.

### CROSS-REFERENCE

This application is a continuation of U.S. patent application Ser. No. 15/294,541, filed on Oct. 14, 2016, which claims the benefit of U.S. Provisional Application No. 62/242,968, filed Oct. 16, 2015, each of which is incorporated herein by reference.

### BACKGROUND OF THE INVENTION

Microscopy is an important tool useful in a variety of clinical and scientific applications including pathology, microbiology, plant tissue culture, animal cell culture, molecular biology, immunology and cell biology. Increasingly important is the acquisition and use of digital images of microscope specimens for digital pathology, where anomalous features in a tissue specimen are located and captured in digital images for analysis. By locating and identifying anomalous features in a tissue specimen, a pathologist can make a diagnosis, help the patient's physician select appropriate treatment and provide information on the efficacy of previous treatments.

### SUMMARY OF THE INVENTION

In general, pathologists often work at locations geographically distant from the hospital or clinic at which a tissue specimen is taken. In the past it was necessary to physically transport a tissue specimen from the location of the patient to the pathologist, for example by express mail or courier. A pathologist would then prepare a slide/specimen from the tissue specimen and examine it under a microscope. However, physically transporting the tissue specimen to the pathology laboratory may be time consuming, particularly if the patient is in a rural or remote area. Furthermore, if the tissue specimen crosses a border, it must be inspected by customs officials. Finally, in many areas such as third world countries there simply are not many pathologists, thereby making it necessary for pathologists to spend an inordinate amount of time travelling to different facilities. For patients who require immediate diagnosis, this is a serious drawback.

The advent of digital pathology helped to alleviate this problem. In digital pathology, a high resolution digital scan of a specimen is taken and this image is electronically transmitted to the pathologist for analysis of the saved image. A physician or technician can prepare slides from tissue specimens and create high resolution scans for off-site analysis by the pathologist. Additionally or alternatively, a pathologist can view and analyze a specimen in real time and then document images of the specimen viewed during analysis. These documented images can then be viewed later by another user for confirmation of an analysis, such as a diagnosis, or for other purposes such as discussion or training.

In digital pathology, a view of a specimen by a digital optical device is often focused prior to acquisition of a digital image of the specimen. In many instances, focusing comprises instructing a digital optical device having a motorized positioning unit to move an entire X-axis or Y-axis of the device up and down or move the optical path up and down until a focused view is obtained. In either case, more movement of the device with the motor is performed than is necessary. By focusing to only the slide or the specimen, there is less strain placed on the motor and unnecessary movement of the device is avoided.

Although digital pathology is an improvement over older pathology methods, it is not without drawbacks. Tissue specimens often have anomalies that require a user to change the depth of focus to view each depth during specimen examination. In traditional microscopy, the different views of the specimen are documented by taking “Z-Stacks” images of varying depth of a specimen and then processing them by either making a three-dimensional object through software analysis, or reassembling an image consisting of only the parts of each image which are determined by software to be in focus, creating an extended depth of focus. However, neither of these processes recreates the experience of viewing through the microscope and many clinical specimens have regions of interest with varying depths. Accordingly, there is a need to document the specimen as it is actually viewed through the microscope by a user during a microscopy session. Additionally, it is of value to accurately and precisely document each and every step which was taken under the microscope to generate the images and make the diagnosis. This allows for enhanced diagnostic accuracy and quality assurance as the diagnosis can be confirmed independently and the methods by which a diagnosis is made can be reviewed. In many instances, a microscopy session is performed by user at a location remote from the microscope.

Another drawback of traditional pathology methods is the “sandbox” approach of viewing a specimen where a user moves the specimen as they please to identify areas of interest. This approach is both inefficient and ineffective, as the user may view multiple regions of the specimen over again as they move the specimen, wasting time and possibly missing an important feature. Thus, there is a need to ensure that a user actually views each region of the specimen that may be of interest. This can be accomplished by defining an area of a specimen to view and moving through the area one field of view at a time based on user defined or predefined intervals until the entirety of the specimen is viewed.

Digital pathology sometimes involves automatic image acquisition of a specimen. This can be accomplished by using a digital optical device to scan and save images of an entire slide or sample. Such a process is ineffective as areas which do not comprise any specimen are acquired, taking up both time and data space. Thus, there is a need for the detection of specimen boundaries. This can be accomplished by the software automatically selecting focus points on a slide or platform comprising a specimen and analyzing each focus point for to determine if the point is within the boundaries of the specimen.

Acquired images of digital pathology are often saved and presented with information regarding the specimen. For example, presentations having specimen images with tissue annotations are used for discussions or tumor boards. Traditionally, these presentations are created by importing the images from one media to a presentation software program and then adding relevant text to the presentation. A method of automatically generating a presentation with acquired images and relevant text, such as image annotation and specimen source information, would save time.

Digital microscopy systems are traditionally designed for use with halogen bulbs, which tend to emit heat in use and can be detrimental to delicate specimens. An alternative approach to specimen illumination involves the use of a light emitting diode (LED) array. An LED array is useful for maintaining sample integrity and can be used for up to tens of thousands of hours without replacement.

In one aspect, disclosed herein is a digital optical device comprising a slide mount for holding a specimen; a motorized positioning unit; a light source; and one or more optical components; wherein the slide mount is positioned along a X-, Y- or Z-axis by the motorized positioning unit and wherein only the slide mount is movable in a Z-axis. In some embodiments, the light source is a halogen bulb. In some embodiments, the light source is a LED array. In some embodiments, the digital optical device is connected to a control computer, wherein the control computer instructs the positioning of the slide mount by the motorizing positioning unit.

In one aspect, disclosed herein are computer-implemented methods of focusing a digital optical device comprising: transmitting, by a computer at a first location, a focusing instruction to a digital optical device at a second location, the focusing instruction comprising one or more commands for the digital optical device to move a slide and a slide mount in a Z-axis to focus a digital optical image; and receiving, by the computer, the focused digital optical image from the digital optical device; provided that the digital optical device moves only the slide and the slide mount in the Z-axis in response to the focusing instruction. In some embodiments, the focusing instruction is sent via a computer network. In some embodiments, the remote digital optical device is a telemicroscope. In some embodiments, the second location is the same location as the first location. In some embodiments, the second location is different from the first location.

In another aspect, disclosed herein are computer-implemented methods of documenting a specimen of interest imaged by a remote digital optical device comprising: transmitting, by a computer at a first location, a first focusing instruction to a digital optical device at a second location different from the first location, the focusing instruction comprising a command for the digital optical device to focus on the top-most plane of an image having a plurality of focus planes; transmitting, by the computer, a second focusing instruction to the digital optical device, the focusing instruction comprising a command for the digital optical device to focus on the bottommost plane of the image; determining, by the computer, a depth of field of the image and an optimal step size based on the depth of field; receiving, by the computer, a sequential series of images, each image created at a different focus plane separated from adjacent planes by the step size; presenting, by the computer, an interface to allow a user at the first location to identify a specimen of interest within the sequential series of images and define a depth of the specimen; and generating, by the computer, a document comprising a plurality of the sequential series of images spanning the depth of the specimen, each image on a separate page of the document. In some embodiments, the focusing instructions are sent via a computer network. In some embodiments, the remote digital optical device is a telemicroscope.

In another aspect, disclosed herein are computer-implemented methods of recording a live viewing history of a specimen evaluated at a digital optical device comprising: receiving, by a computer at a first location, one or more micrographs of the specimen, the one or more micrographs generated by a digital optical device at a second location; receiving, by the computer, a plurality of data describing a live viewing session of the specimen at the remote digital optical device, the plurality of data comprising X- and Y-position of stage, focus, and magnification of the remote digital optical device captured repetitively at a time interval; generating, by the computer, a live viewing history from the plurality of data; and applying, by the computer, the live viewing history to the one or more micrographs of the specimen to output a video file that replicates the live viewing session. In some embodiments, the time interval is user-defined. In some embodiments, the method further comprises presenting, by the computer, an interface allowing a user at the first location to record voice audio and wherein the outputting of the video file comprises integrating the voice audio into the video file. In some embodiments, the method further comprises comparing, by the computer, a total tissue of the specimen to tissue viewed in the live viewing session to generate a score. In some embodiments, the method further comprises: creating a vector trail of the X- and Y-position of stage and focus of the remote digital optical device relative to the specimen; and overlaying the vector trail on the one or more micrographs of the specimen. In some embodiments, the second location is the same location as the first location. In some embodiments, the second location is different from the first location.

In another aspect, disclosed herein are computer-implemented methods of evaluating a specimen at a digital optical device comprising: receiving, by a computer at a first location, one or more micrographs of the specimen as a live stream of constantly refreshing images, the one or more micrographs generated by a digital optical device at a second location as a live stream of constantly refreshing images; presenting, by the computer, an interface allowing a user at the first location to define a total viewing area for the specimen; separating, by the computer, the total viewing area into a plurality of fields of view; and transmitting, by the computer, instructions to the remote digital optical device, the instructions comprising one or more commands for the digital optical device to move a stage of the device to advance through the fields of view at a repeating time interval. In some embodiments, the time interval is user-defined. In some embodiments, the instructions comprise one or more commands for the digital optical device to move the stage to advance through the fields of view in a pattern corresponding to rows across the total viewing area. In some embodiments, the instructions comprise one or more commands for the digital optical device to move the stage to advance through the fields of view in a pattern corresponding to columns across the total viewing area or a straight line. In some embodiments, the method further comprises automatically determining the area of a total of tissue detected in the specimen. In some embodiments, the second location is the same location as the first location. In some embodiments, the second location is different from the first location. In some embodiments, a desktop application is implemented by the computer at the first location by the user to evaluate the specimen.

In another aspect, disclosed herein are computer-implemented methods of automatically generating a presentation on the evaluation of a specimen at a digital optical device comprising: receiving, by a computer at a first location, a color preview micrograph of the specimen, the preview micrograph generated by a digital optical device at a second location; performing, by the computer, a white balance on the preview micrograph; determining, by the computer, the dominant colors in the preview micrograph; defining, by the computer, an area to scan based on detection of a contiguous region of the preview micrograph including the dominant colors; generating, by the computer, a plurality of focus points within the area to scan; evaluating, by the computer, each focus point to determine if it is above tissue of the specimen based on the dominant colors; and adjusting, by the computer, the position of any focus points that are not over tissue of the specimen and repeating the evaluation. In some embodiments, the determining of the dominant colors in the preview micrograph comprises determining a modal value of the colors and subsequently applying a white threshold, a black threshold, a color threshold, and a paleness threshold. In some embodiments, the second location is the same location as the first location. In some embodiments, the second location is different from the first location.

In another aspect, disclosed herein are computer-implemented methods of automatically generating a presentation or report on the evaluation of a specimen at a digital optical device comprising: storing, by a computer at a first location, one or more presentation templates; receiving, by the computer, a color preview micrograph of the specimen, the preview micrograph generated by a digital optical device at a second location; receiving, by the computer, one or more high magnification micrographs of the specimen, the one or more high-magnification micrographs optionally associated with text annotation; and generating, by the computer, the presentation by integrating the color preview micrograph of the specimen and the one or more high-magnification micrographs into a selected presentation template, the presentation comprising the color preview micrograph, the color preview micrograph linked to the one or more high-magnification micrographs and the associated text annotations, if any, the links indicating the position within the specimen each high-magnification micrograph was created. In some embodiments, the method further comprises presenting an interface allowing a user at the first location to integrate one or more previously generated presentations into the presentation. In some embodiments, the second location is the same location as the first location. In some embodiments, the second location is different from the first location.

In another aspect, disclosed herein are computer-implemented methods of illuminating a specimen within a digital optical device comprising positioning an LED array on the side of the specimen opposite an imaging mechanism of the digital optical device, and placing a holographic light diffusing substrate between the LED array and the specimen.

In another aspect, disclosed herein are digital optical devices comprising: an electromagnet; a stage; and a specimen eject mechanism; the electromagnet configured to fix position of the stage when the specimen eject mechanism is activated. In some embodiments, the digital optical device is a microscope. In further embodiments, the microscope is a remotely operated telemicroscope. In further embodiments, the device is a whole slide imaging scanner.

In another aspect, disclosed herein are digital optical devices comprising: a memory; an optical array; a stage; a digital image capture unit; and a motorized positioning unit; the X-, Y-, and Z-positions of the optical array relative to the stage stored in the memory upon each activation of the digital image capture unit; the motorized positioning unit configured to return the optical array to the recorded positions associated with a particular digital image upon request from a user. The Y position also reports which slide among multiple slides is being viewed. In some embodiments, the digital optical device is a microscope. In further embodiments, the microscope is a remotely operated telemicroscope.

In another aspect, disclosed herein are computer-implemented systems for telemicroscopy comprising: (a) a digital optical device comprising a slide mount having a range of Z focus between a first position and a second position; a motor for moving the slide mount within the Z focus range; a light source; and an optical component; (b) a digital processing device comprising at least one processor, an operating system configured to perform executable instructions, a memory, and a computer program including instructions executable by the digital processing device to create a telemicroscopy focus application comprising: a software module instructing the motor of the digital optical device to move in a Z-axis in a number of steps between the first position and the second position to focus through a digital optical image; and a software module receiving the focusable digital optical image from the digital optical image device; wherein the digital processing device and digital optical device send and receive instructions, respectively, over a telecommunication network. In some embodiments, the digital optical device comprises an imaging device and wherein the application comprises a software module instructing the imaging device to acquire a micrograph of the focused digital optical image. In some embodiments, the application comprises a software module instructing the digital optical device to import the acquired micrograph into a presentation. In some embodiments, the light source is a LED and the optical component is a light shaping diffuser. In some embodiments, the digital optical device is located at a first location and the digital processing device is located at a second location different from the first location.

In another aspect, disclosed herein are non-transitory computer-readable storage media encoded with a computer program including instructions executable by a processor to create an application for focusing a digital optical device, the application comprising a software module instructing a motor of the digital optical device to move along a Z-axis a fixed number of steps between a first position and a second position to create a focusable digital optical image; and a software module receiving a focused digital optical image from the digital optical image device; wherein the digital processing device and digital optical device send and receive instructions, respectively, over a telecommunication network. In some embodiments, the application further comprises a software module instructing an imaging device operably connected to the digital optical device to acquire a micrograph of the focusable digital optical image. In some embodiments, the digital optical device is located at a first location and the digital processing device is located at a second location different from the first location.

In another aspect, disclosed herein are computer-implemented systems for telemicroscopy comprising: (a) a digital optical device comprising a slide mount having a range of Z focus between a first position and a second position; a motor for moving the slide mount within the Z focus range; a light source; and an optical component; (b) a digital processing device comprising at least one processor, an operating system configured to perform executable instructions, a memory, and a computer program including instructions executable by the digital processing device to create a telemicroscopy focus application comprising: a software module instructing the motor of the digital optical device to move in a Z-axis between the first position and the second position to focus on the top-most plane of an image having a plurality of focus planes; a software module instructing the motor of the digital optical device to move in a Z-axis between the first position and the second position to focus on the bottom-most plane of the image; a software module determining a depth of field of the image and an optimal step size based on the depth of field; a software module receiving a sequential series of images, each image created at a different focus plane separated from adjacent planes by the step size; a software module presenting an interface to allow a user to identify a specimen of interest within the sequential series of images and define a depth of the specimen; and a software module generating a document comprising a plurality of the sequential series of images spanning the depth of the specimen, each image on a separate page of the document; wherein the digital processing device and digital optical device send and receive instructions, respectively, over a telecommunication network. In some embodiments, the light source is a LED and the optical component is a light shaping diffuser. In some embodiments, the digital optical device is located at a first location and the digital processing device and user are located at a second location different from the first location.

In another aspect, disclosed herein are non-transitory computer-readable storage media encoded with a computer program including instructions executable by a processor to create an application for documenting a series of images with a digital optical device, the application comprising a software module instructing a motor of the digital optical device to move in a Z-axis between a first position and a second position to focus on the top-most plane of an image having a plurality of focus planes; a software module instructing the motor of the digital optical device to move in a Z-axis between the first position and the second position to focus on the bottom-most plane of the image; a software module determining a depth of field of the image and an optimal step size based on the depth of field; a software module receiving a sequential series of images, each image created at a different focus plane separated from adjacent planes by the step size; a software module presenting an interface to allow a user to identify a specimen of interest within the sequential series of images and define a depth of the specimen; and a software module generating a document comprising a plurality of the sequential series of images spanning the depth of the specimen, each image on a separate page of the document; wherein the digital processing device and digital optical device send and receive instructions, respectively, over a telecommunication network. In some embodiments, the application further comprises a software module instructing the digital optical device to import one or more of the series of images into a presentation. In some embodiments, the digital optical device is located at a first location and the digital processing device and user are located at a second location different from the first location.

In another aspect, disclosed herein are computer-implemented systems for telemicroscopy comprising: (a) a digital optical device comprising a slide mount having a range of positions along a X- and Y-axis; a motor for moving the slide mount along the X- and Y-axis; a light source; and an optical component; (b) a digital processing device comprising at least one processor, an operating system configured to perform executable instructions, a memory, and a computer program including instructions executable by the digital processing device to create an application for recording a telemicroscopy viewing history comprising: a software module receiving one or more micrographs of a specimen positioned on the slide mount of the digital optical device; a software module receiving a plurality of data describing a live viewing session of the specimen, the plurality of data comprising X- and Y-position of the slide mount, focus, and magnification of the digital optical device captured and the time at which the changed event occurred, a software module generating a live viewing history from the plurality of data; In some embodiments, a software module applying the live viewing history to the one or more micrographs of the specimen to output a video file that replicates the live viewing session; wherein the digital optical device and digital processing device send and receive micrographs and data, respectively, over a telecommunication network. In some embodiments, the light source is a LED and the optical component is a light shaping diffuser. In some embodiments, the application further comprises a software module instructing the digital optical device to import the video file into a presentation. In some embodiments, the time interval is user-defined. In some embodiments, the time interval matches exactly to the viewing history of the original user. In some embodiments, the application further comprises a software module presenting an interface allowing a user to record voice audio and wherein the outputting of the video file comprises integrating the voice audio into the video file. In some embodiments, the application further comprises a software module comparing a total tissue of the specimen to tissue viewed in the live viewing session to generate a score. In some embodiments, the application further comprises creating a vector trail of the X- and Y-positions of slide mount and focus of the digital optical device relative to the specimen; and overlaying the vector trail on the one or more micrographs of the specimen. In some embodiments, the digital optical device is located at a first location and the digital processing device is located at a second location different from the first location.

In another aspect, disclosed herein are non-transitory computer-readable storage media encoded with a computer program including instructions executable by a processor to create an application for recording a live viewing history of a specimen evaluated with a digital optical device, the application comprising a software module receiving one or more micrographs of a specimen positioned on the slide mount of the digital optical device; a software module receiving a plurality of data describing a live viewing session of the specimen, the plurality of data comprising X- and Y position of the slide mount, focus, and magnification of the digital optical device captured repetitively and a time stamp; a software module generating a live viewing history from the plurality of data; and a software module applying the live viewing history to the one or more micrographs of the specimen to output a video file that replicates the live viewing session; wherein the digital optical device and digital processing device send and receive micrographs and data, respectively, over a telecommunication network. In some embodiments, the application further comprises a software module instructing the digital optical device to import the video file into a presentation. In some embodiments, the time interval is user-defined. In some embodiments, the application further comprises a software module presenting an interface allowing a user to record voice audio and wherein the outputting of the video file comprises integrating the voice audio into the video file. In some embodiments, the application further comprises a software module comparing a total tissue of the specimen to tissue viewed in the live viewing session to generate a score. In some embodiments, the application further comprises creating a vector trail of the X- and Y-positions of slide mount and focus of the digital optical device relative to the specimen; and overlaying the vector trail on the one or more micrographs of the specimen. In some embodiments, wherein the digital optical device is located at a first location and the digital processing device is located at a second location different from the first location.

In another aspect, described herein are computer-implemented systems for telemicroscopy comprising: (a) a digital optical device comprising a slide mount having a range of positions along a X- and Y-axis defining fields of view of a specimen positioned on the slide mount; a motor for moving the slide mount along the X- and Y-axis; a light source; and an optical component; (b) a digital processing device comprising at least one processor, an operating system configured to perform executable instructions, a memory, and a computer program including instructions executable by the digital processing device to create a specimen evaluation application comprising: a software module receiving one or more micrographs of the specimen, the one or more micrographs generated by the digital optical device; a software module presenting an interface allowing a user to define a total viewing area for the specimen; a software module separating the total viewing area into a plurality of fields of view; and a software module instructing the motor to move the slide mount to advance through the fields of view at a repeating time interval; wherein the digital optical device and digital processing device send and receive information over a telecommunication network. In some embodiments, the light source is a LED and the optical component is a light shaping diffuser. In some embodiments, the time interval is user-defined. In some embodiments, the instructions comprise one or more commands for the motor to move the slide mount to advance through the fields of view in a pattern corresponding to rows across the total viewing area. In some embodiments, the instructions comprise one or more commands for the motor to move the slide mount to advance through the fields of view in a pattern corresponding to columns across the total viewing area. In some embodiments, the application further comprises a software module automatically determining the area of a total of tissue detected in the specimen. In some embodiments, the instructions comprise one or more commands for the motor to move the slide mount to advance through the fields of view in a straight line between two user defined points. In some embodiments, the digital optical device is located at a first location and the digital processing device and user are located at a second location different from the first location.

In another aspect, disclosed herein are non-transitory computer-readable storage media encoded with a computer program including instructions executable by a processor to create an application for evaluating a specimen with a digital optical device, the application comprising a software module receiving one or more micrographs of the specimen, the one or more micrographs generated by the digital optical device; a software module presenting an interface allowing a user to define a total viewing area for the specimen; a software module separating the total viewing area into a plurality of fields of view; and a software module instructing the motor to move the slide mount to advance through the fields of view at a repeating time interval; wherein the digital optical device and digital processing device send and receive information over a telecommunication network. In some embodiments, the light source is a LED and the optical component is a light shaping diffuser. In some embodiments, the time interval is user-defined. In some embodiments, the instructions comprise one or more commands for the motor to move the slide mount to advance through the fields of view in a pattern corresponding to rows across the total viewing area. In some embodiments, the instructions comprise one or more commands for the motor to move the slide mount to advance through the fields of view in a pattern corresponding to columns across the total viewing area. In some embodiments, the instructions comprise one or more commands for the motor to move the slide mount to advance through the fields of view in a straight line between two user defined points. In some embodiments, the application further comprises a software module automatically determining the area of a total of tissue detected in the specimen. In some embodiments, the digital optical device is located at a first location and the digital processing device and user are located at a second location different from the first location.

In another aspect, disclosed herein are computer-implemented systems for telemicroscopy comprising: (a) a digital optical device comprising a slide mount for holding a specimen and a scanning imaging device; (b) a digital processing device comprising at least one processor, an operating system configured to perform executable instructions, a memory, computer program including instructions executable by the digital processing device to create a presentation application comprising: a software module receiving a color preview micrograph of the specimen, the preview micrograph generated by the digital optical device; a software module performing a white balance on the preview micrograph; a software module determining the dominant colors in the preview micrograph; a software module defining an area to scan based on detection of a contiguous region of the preview micrograph including the dominant colors; a software module generating a plurality of focus points within the area to scan; a software module evaluating each focus point to determine if it is above tissue of the specimen based on the dominant colors; and a software module adjusting the position of any focus points that are not over tissue of the specimen and repeating the evaluation; wherein the digital processing device receives the preview micrograph over a telecommunication network. In some embodiments, the determining of the dominant colors in the preview micrograph comprises determining a modal value of the colors and subsequently applying a white threshold, a black threshold, a color threshold, and a paleness threshold. In some embodiments, the digital optical device is located at a first location and the digital processing device is located at a second location different from the first location.

In another aspect, disclosed herein are non-transitory computer-readable storage media encoded with a computer program including instructions executable by a processor to create an application for automatically generating a presentation on the evaluation of a specimen at a digital optical device, the application comprising a software module receiving a color preview micrograph of the specimen, the preview micrograph generated by the digital optical device; a software module performing a white balance on the preview micrograph; a software module determining the dominant colors in the preview micrograph; a software module defining an area to scan based on detection of a contiguous region of the preview micrograph including the dominant colors; a software module generating a plurality of focus points within the area to scan; a software module evaluating each focus point to determine if it is above tissue of the specimen based on the dominant colors; and a software module adjusting the position of any focus points that are not over tissue of the specimen and repeating the evaluation; the digital processing device receives the preview micrograph over a telecommunication network. In some embodiments, the determining of the dominant colors in the preview micrograph comprises determining a modal value of the colors and subsequently applying a white threshold, a black threshold, a color threshold, paleness threshold. In some embodiments, the digital optical device is located at a first location and the digital processing device is located at a second location different from the first location.

In another aspect, disclosed herein are computer-implemented systems for telemicroscopy comprising: (a) a digital optical device comprising a slide mount for holding a specimen and a scanning imaging device; (b) a digital processing device comprising at least one processor, an operating system configured to perform executable instructions, a memory, and a computer program including instructions executable by the digital processing device to create a presentation application comprising: a software module storing one or more presentation templates; a software module receiving a color preview micrograph of the specimen, the preview micrograph generated by the digital optical device; a software module receiving one or more high-magnification micrographs of the specimen, the one or more high-magnification micrographs optionally associated with text annotation; and a software module generating the presentation by integrating the color preview micrograph of the specimen and the one or more high-magnification micrographs into a selected presentation template, the presentation comprising the color preview micrograph, the color preview micrograph linked to the one or more high-magnification micrographs and the associated text annotations, if any, the links indicating the position within the specimen each high-magnification micrograph was created; wherein the digital processing device receives the preview and highmagnification micrographs over a telecommunication network. In some embodiments, the application further comprises a software module presenting an interface allowing a user to integrate one or more previously generated presentations into the presentation. In some embodiments, the digital optical device is located at a first location and the digital processing device is located at a second location different from the first location.

In another aspect, disclosed herein are non-transitory computer-readable storage media encoded with a computer program including instructions executable by a processor to create an application for automatically generating a presentation on the evaluation of a specimen at a digital optical device, the application comprising a database comprising one or more presentation templates; a software module storing one or more presentation templates; a software module receiving a color preview micrograph of the specimen, the preview micrograph generated by the digital optical device; a software module receiving one or more high-magnification micrographs of the specimen, the one or more high-magnification micrographs optionally associated with text annotation; software module generating the presentation by integrating the color preview micrograph of the specimen and the one or more high-magnification micrographs into a selected presentation template, the presentation comprising the color preview micrograph, the color preview micrograph linked to the one or more high-magnification micrographs and the associated text annotations, if any, the links indicating the position within the specimen each high-magnification micrograph was created; wherein the digital processing device receives the preview and high-magnification micrographs over a telecommunication network. In some embodiments, the application further comprises a software module presenting an interface allowing a user to integrate one or more previously generated presentations into the presentation. In some embodiments, the digital optical device is located at a first location and the digital processing device is located at a second location different from the first location.

### BRIEF DESCRIPTION OF THE DRAWINGS

FIG. 1 shows a non-limiting example of a digital optical device having a slide only focus.

FIG. 2 shows a non-limiting example of the data acquisition flow of the positional recording process and some potential playback options. Additional playback options are able to be integrated and devised based on the positional data recorded.

FIG. 3 shows a non-limiting example of a slide with an area selected and a highlighted example traverse pattern which can be programmed to view by row or by column.

FIG. 4 shows an exemplary computer-implemented method for identifying a tissue on a stage of a digital optical device.

FIG. 5 shows a non-limiting example of the LED illumination system with holographic light shaping diffuser as it relates to the specimen.

FIG. 6 shows a non-limiting example of a digital optical device comprising an electromagnet.

FIG. 7 shows components of an exemplary digital optical device comprising a halogen bulb light source.

FIG. 8 shows components of an exemplary digital optical device comprising a LED array light source.

FIG. 9 shows a slide of a presentation automatically generated with images of a specimen acquired using a digital optical device described herein, specimen source information, and a case summary.

FIG. 10 shows a slide of a presentation automatically generated with a low resolution image of a specimen and high resolution images of 10 distinct regions of the specimen mapped on the high resolution image.

FIG. 11 shows a slide of a presentation automatically generated with a high resolution image of a specimen acquired using a digital optical device described herein and annotations made by a user during specimen viewing.

### DETAILED DESCRIPTION OF THE INVENTION

Current digital pathology methods rely on the documentation of isolated images of specimens that do not accurately convey the complexity of the specimen, making it difficult to reproduce or understand how an analysis of the specimen was performed from the static images. There is a need for accurate documentation of digital microscopy specimens that allow for a user to view and analyze the saved images of a specimen as though the user is viewing the specimen under a microscope. Wherein the digital images are acquired for diagnostic purposes, there is also a need for documenting each and every step which was taken under the microscope to arrive at the documented images, so that these steps may be repeated for clinical, research, or educational purposes. The present disclosure, in various embodiments, describes methods of enhanced digital microscopy for the acquisition and documentation of microscopy specimens.

Described herein, in certain embodiments are computer-implemented methods of focusing a digital optical device comprising: transmitting, by a computer at a first location, a focusing instruction to a digital optical device at a second location different from the first location, the focusing instruction comprising one or more commands for the digital optical device to move a slide and a slide mount in a Z-axis to focus a digital optical image; and receiving, by the computer, the focused digital optical image from the digital optical device; provided that the digital optical device moves only the slide and the slide mount in the Z-axis in response to the focusing instruction.

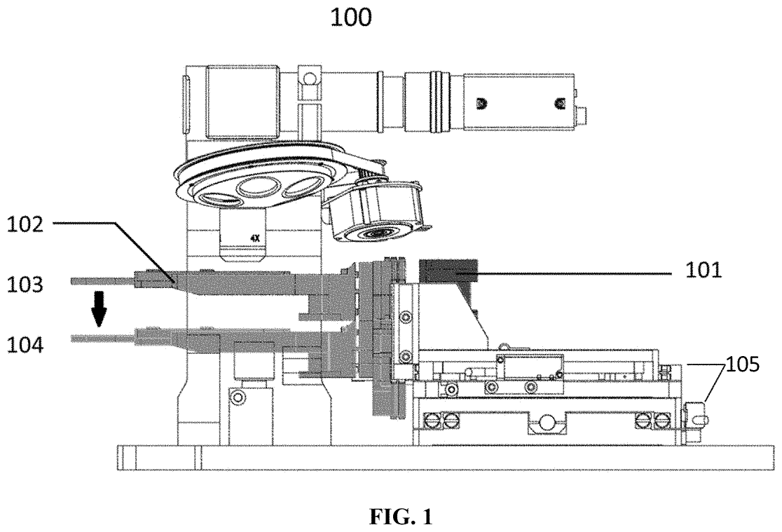

Further described herein is a digital optical device comprising one or more optical components and a slide mount, wherein the slide mount is the only component of the device that is movable in a Z-axis. An example of such digital optical device is shown as device 100 in FIG. 1.

Two end positions indicating a range of Z focus 101 for slide mount 102 are shown in a first position 103 and a second position 104. The focusing axis is affixed to the top of the X/Y stage 105. In this device, the focusing element does not need to support the weight or mechanisms of X or Y axis or of the nosepiece or optical components.

Also described herein, in certain embodiments are computer-implemented methods of documenting specimen of interest imaged by a digital optical device comprising: transmitting, by a computer at a first location, a first focusing instruction to a digital optical device at a second location, the focusing instruction comprising a command for the digital optical device to focus on the top-most plane of an image having a plurality of focus planes; transmitting, by the computer, a second focusing instruction to the digital optical device, the focusing instruction comprising a command for the digital optical device to focus on the bottom-most plane of the image; determining, by the computer, a depth of field of the image and an optimal step size based on the depth of field; receiving, by the computer, a sequential series of images, each image created at a different focus plane separated from adjacent planes by the step size; presenting, by the computer, an interface to allow a user at the first location to identify a specimen of interest within the sequential series of images and define a depth of the specimen; and generating, by the computer, a document comprising a plurality of the sequential series of images spanning the depth of the specimen, each image on a separate page of the document. In some embodiments, the second location is the same location as the first location. In some embodiments, the second location is different from the first location.

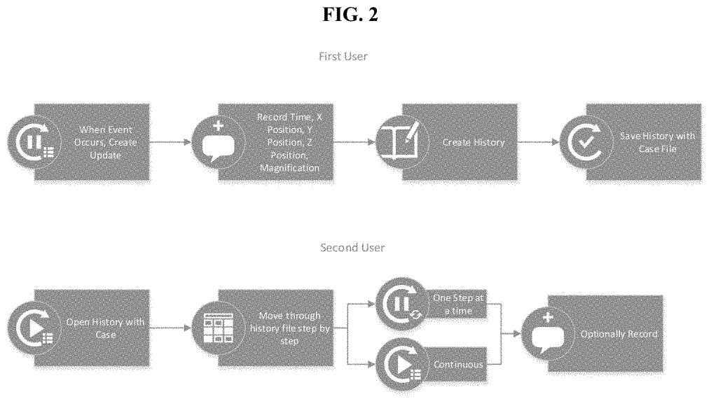

Also described herein, in certain embodiments are computer-implemented methods of recording a live viewing history of a specimen evaluated at a digital optical device comprising: receiving, by a computer at a first location, one or more micrographs of the specimen, the one or more micrographs generated by a digital optical device at a second location; receiving, by the computer, a plurality of data describing a live viewing session of the specimen at the digital optical device, the plurality of data comprising X- and Y-position of stage, focus, and magnification of the digital optical device captured repetitively at a time interval; generating, by the computer, a live viewing history from the plurality of data; and applying, by the computer, the live viewing history to the one or more micrographs of the specimen to output a video file that replicates the live viewing session. In some embodiments, the second location is the same location as the first location. In some embodiments, the second location is different from the first location. An exemplary process workflow for a method of recording a live viewing history is shown in FIG. 2. Referring to the first panel of FIG. 2, a first user views a specimen positioned on a slide stage of a digital optical device in real time. The first user is at a location remote from the device and views the specimen using a remote viewing station. The user controls the optical device using a remote computer of the remote viewing station that is connected to the optical device via a computer network. The user instructs the device via the remote computer to move the slide mount until an area of interest of the specimen is viewable by the first user. The remote computer moves the slide mount of the optical device in X- and Y-axes to identify the area of interest, and focuses a view of the area by moving the slide stage in a Z-axis. The user may also instruct the optical device, via the remote computer, to change an objective lens of the device during focusing. A micrograph of the area of interest in view to the first user is recorded when the first user instructs an imaging device operably connected to the optical device to acquire and store said micrograph. The X, Y and Z positions of the slide stage that correspond to the micrograph is recorded. Also recorded is the magnification used and time of micrograph acquisition. The first user optionally repeats this process so that a plurality of micrographs with corresponding data is recorded and stored. In some cases, the first user instructs a computer to record a series of micrographs and corresponding data during all or a portion of the time that the first user is viewing the specimen. In some cases, the first user instructs a computer to record data at regular intervals during a viewing session. For example, data is recorded every few milliseconds, seconds or minutes of a viewing session. As another example, data is recorded when a user identifies a new area of interest of the specimen. In some cases, the first user instructs a computer to record data continuously for a given period of time during a viewing session. A history file is created with the recorded values. The history file is then saved with a casefile comprising specimen information. In some embodiments, the micrograph and corresponding data are recorded on any device of the computer network. For example, the computer network comprises a server and the micrograph and corresponding coordinate data and viewing history are stored on the server.

Referring to the second panel of FIG. 2, a second user opens the stored history of the specimen using a viewing computer. In some cases, the viewing computer is a computer independent of the computer network. In other cases, the viewing computer is a computer connected to the computer network. The second user moves through the history file in either a stepwise or continuous manner. In a stepwise method, the second user views each recorded micrograph at a defined period of time, and manually instructs the computer to move through each micrograph. In a continuous method, the second user views the recorded micrographs as a video, where the second user can optionally control the speed of the video, as well as pause the video. In some embodiments, the second user views the specimen in real time by instructing a computer to position the specimen using the recorded X, Y, and Z coordinates. In this method, the second user views the same areas of the specimen, at the same focal points, as the first user. In some methods, the viewing history of the second user is recorded. An example of a text output from the workflow of FIG. 2 is shown below.

```
<XYZMagHistory>
  <Entry>
    <Time>2015-10-07_01-10-31-397</Time>
    <Magnification>2x</Magnification>
    <X>12.7</X>
    <Y>25.4</Y>
    <Z>25.4</Z>
  </Entry>
  <Entry>
    <Time>2015-10-07_01-10-33-964</Time>
    <Magnification>2x</Magnification>
    <X>18.965318399999994</X>
    <Y>37.507303799999995</Y>
    <Z>37.5073038</Z>
  </Entry>
  <Entry>
    <Time>2015-10-07_01-10-36-459</Time>
    <Magnification>2x</Magnification>
    <X>17.965318399999994</X>
    <Y>37.507303799999995</Y>
    <Z>37.5073038</Z>
  </Entry>
```

In some embodiments, micrographs recorded by the first user are displayed as an overlay to real time views of the specimen by the second user.

In some embodiments, micrographs of the specimen recorded by the first user are displayed in a three-dimensional surface map comprising a plurality of vectors, wherein each vector correlates to a micrograph recorded at a specific stage position and focus.

In some embodiments, the first user instructs a computer to scan over the entire area of a slide on which the specimen is positioned. The first user then views and records micrographs of defined regions of the specimen. The first user instructs the computer to save coordinates of the defined regions viewed and regions not viewed in the file history. The second user can load the specimen on the optical device used by the first user, and upload the specimen file history on a computer. The second user then instructs the computer used by the second user to position the specimen at coordinates that were not viewed by the first user.

In some embodiments, a method of recording a live viewing history as described is annotated with a voice recording that is synchronized in time with the data recorded. In some embodiments, a second user views a video file stored by the first user and hears an audio file of a voice recording that is synchronized with the video.

Also described herein, in certain embodiments are computer-implemented methods of evaluating a specimen at a digital optical device comprising: receiving, by a computer at a first location, one or more micrographs of the specimen, the one or more micrographs generated by a digital optical device at a second location; presenting, by the computer, an interface allowing a user at the first location to define a total viewing area for the specimen; separating, by the computer, the total viewing area into a plurality of fields of view; and transmitting, by the computer, instructions to the remote digital optical device, the instructions comprising one or more commands for the digital optical device to move a stage of the device to advance through the fields of view at a repeating time interval. In some embodiments, the second location is the same location as the first location. In some embodiments, the second location is different from the first location.

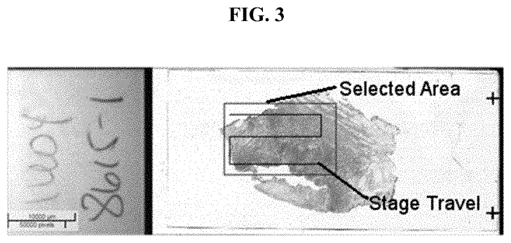

An example pattern that defines different fields of view of a specimen is shown in FIG. 3. The user remotely views the specimen and identifies an area of interest for further analysis. Alternatively, a computer scans the specimen and identifies an area of interest for further analysis. In FIG. 3, the area of interest is designated by a box (“selected area”). The user or computer determines a pattern for viewing the specimen. In FIG. 3, the pattern of stage travel is indicated. In some cases, the user instructs a computer controlling the digital optical device to advance fields of view in the pattern over a defined course of time. In some cases, the user instructs a computer controlling the digital optical device to advance the field of view manually, so that the user may view the specimen for any amount of time desirable before advancing to a new field of view.

Also described herein, in certain embodiments are computer-implemented methods of automatically generating a presentation on the evaluation of a specimen at a digital optical device comprising: receiving, by a computer at a first location, a color preview micrograph of the specimen, the preview micrograph generated by a digital optical device at a second location; performing, by the computer, a white balance on the preview micrograph; determining, by the computer, the dominant colors in the preview micrograph; defining, by the computer, an area to scan based on detection of a contiguous region of the preview micrograph including the dominant colors; generating, by the computer, a plurality of focus points within the area to scan; evaluating, by the computer, each focus point to determine if it is above tissue of the specimen based on the dominant colors; and adjusting, by the computer, the position of any focus points that are not over tissue of the specimen and repeating the evaluation. In some embodiments, the second location is the same location as the first location. In some embodiments, the second location is different from the first location.

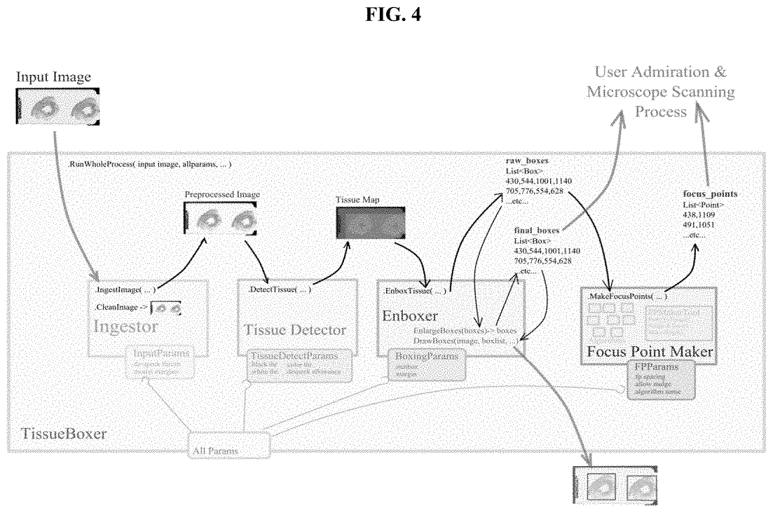

An exemplary method for evaluating boundaries of a specimen is shown in the workflow of FIG. 4. The method of FIG. 4 has four main computer-implemented steps: (1) ingest image; (2) detect tissue; (3) enbox tissue blobs; and (4) generate anchor points. For the first step, a Bitmap of a preview micrograph is received from a digital optical device and the following actions are performed: white balance on the preview micrograph; determining dominant colors in the preview micrograph; erasing small dark specs from the preview micrograph; and reducing micrograph size for fast processing. The output is a cleaned, reduced-size micrograph that is input into the second method step. In the second step, the following actions are performed on the output from step 1: detecting all pixels that are too dark (“black”) and are therefore non-tissue; detecting all pixels that are too bright (“white”) and are therefore assumed to be background and therefore non-tissue; detecting pixels with sufficient color to be declared as “tissue”; and cleaning a resulting map of tissue of any small specks, typically due to noise or iridescence along label or cover slip edges. The output is a tissue map in a Bitmap that is input into step 3. Step 3 of the method comprises the following actions performed on the tissue map: identifying areas of tissue surrounded by background; defining a box around each area; merging overlapping boxes; and eliminating boxes too small to be plausible as tissue. The output is a list of raw boxes, where each box tightly surrounds a tissue area, or an interlocking set of tissue areas. In the step 4 of the method, the input is a tissue map and a list of raw boxes, where the following actions are performed for each raw box: identifying the box center, corners, and side mid-points; defining proposed focus points; and adjusting proposed focus points so that a focus point is not located on a tissue hole or crack. The output from step 4 is a list comprising X and Y coordinates of a tissue suitable for drawing marks on a display screen or instructing a microscope stage to move to said coordinates.

Also described herein, in certain embodiments are computer-implemented methods of automatically generating a presentation on the evaluation of a specimen at a digital optical device comprising: storing, by a computer at a first location, one or more presentation templates; receiving, by the computer, a color preview micrograph of the specimen, the preview micrograph generated by a digital optical device at a second location; receiving, by the computer, one or more highmagnification micrographs of the specimen, the one or more high-magnification micrographs optionally associated with text annotation; and generating, by the computer, the presentation by integrating the color preview micrograph of the specimen and the one or more high-magnification micrographs into a selected presentation template, the presentation comprising the color preview micrograph, the color preview micrograph linked to the one or more high-magnification micrographs and the associated text annotations, if any, the links indicating the position within the specimen each high-magnification micrograph was created. In some embodiments, the second location is the same location as the first location. In some embodiments, the second location is different from the first location.

Also described herein, in certain embodiments are computer-implemented methods of illuminating a specimen within a digital optical device comprising positioning an LED array on the side of the specimen opposite an imaging mechanism of the digital optical device, and placing a holographic light diffusing substrate between the LED array and the specimen.

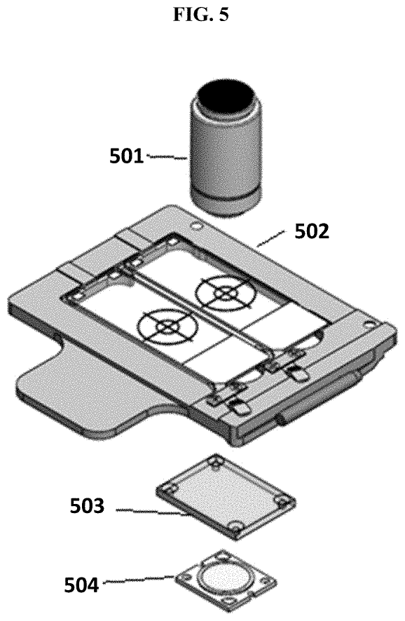

An exemplary embodiment of an LED illumination system useful in a digital optical device and microscopy methods described herein is shown in FIG. 5. The LED illumination system of FIG. 5 comprises an objective lens 501, a slide holder 502, a light shaping diffuser 503 and an LED illuminator 504.

Also described herein, in certain embodiments are digital optical devices comprising: an electromagnet; a stage; and a specimen eject mechanism; the electromagnet configured to fix position of the stage when the specimen eject mechanism is activated.

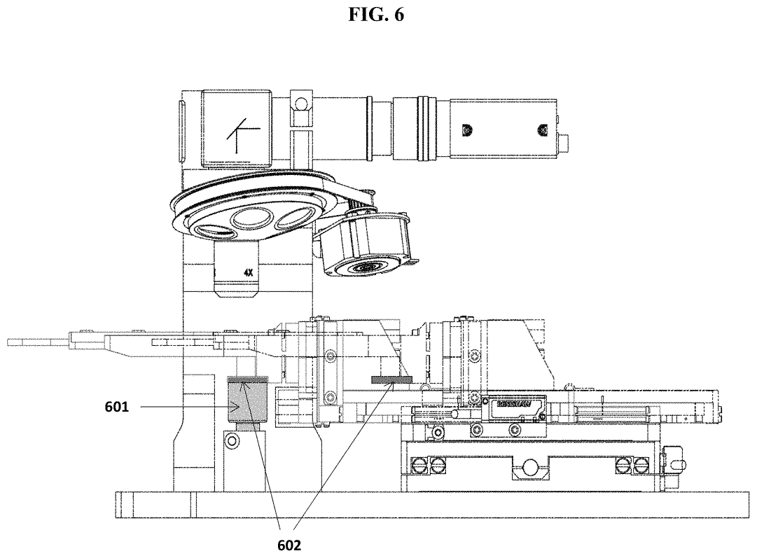

An exemplary embodiment of a digital optical device comprising an electromagnet is shown in FIG. 6. Electromagnet 601 is controlled with a magnetically attractive metal cap 602. In the device of FIG. 6, metal cap 602 is movable between two positions: an in and out position. The out position is the position of the cap when the stage is positioned for specimen viewing. The in position is the position of the cap when the stage is moved outward from the device for specimen loading and unloading. When the cap is in the out position, the electromagnet is not powered. When the system moves into loaded and unloading position (“in” position), the electromagnet activates, locking the cap into a secure position, and securing the stage from unwanted motion during loading or unloading of the specimen.

Also described herein, in certain embodiments are digital optical devices comprising: a memory; an optical array; a stage; a digital image capture unit; and a motorized positioning unit; the X-, Y-, and Z-positions of the optical array relative to the stage stored in the memory upon each activation of the digital image capture unit; the motorized positioning unit configured to return the optical array to the recorded positions associated with a particular digital image upon request from a user.

### Certain Definitions

Unless otherwise defined, all technical terms used herein have the same meaning as commonly understood by one of ordinary skill in the art to which this invention belongs. As used in this specification and the appended claims, the singular forms “a, an,” and “the” include plural references unless the context clearly dictates otherwise. Any reference to “or” herein is intended to encompass “and/or” unless otherwise stated.

### Digital Optical Device

A digital optical device includes, without limitation, a microscope and components thereof useful for viewing a specimen. In some embodiments, a digital optical device comprises a slide mount for holding a specimen and/or slide comprising a specimen. In some embodiments, a digital optical device comprises a light source such as a halogen bulb and one or more optical components, such as a condenser and objective lens. In some embodiments, a digital optical device comprises an LED array and a holographic light diffusing substrate.

In some embodiments, a specimen presented on a slide is viewed with a digital optical device by moving the slide and slide mount in a Z-axis to focus a view of the specimen. In some cases, no other component of the digital optical device is moved in the Z-axis during the focusing. For example, the digital optical device is a microscope comprising one or more optical components that are not moved in the Z-axis during focusing.

A digital optical device is configured with or comprises a digital acquisition device is configured to acquire one or more images of a specimen. In some embodiments, the digital acquisition device is a camera. In some embodiments, the camera is a low magnification camera. Examples of an acquisition device include, without limitation, a CCD and linear array.

In some embodiments, an acquired image of the specimen is saved to a storage system and/or displayed, wherein the images displayed can be saved images, live images or both saved and live images of the specimen. A live image, in many instances, refers to an image of a sample present in the system at the same time the image is being displayed, allowing for the live control of the view of said image.

In some embodiments, the digital optical device is integrated with a computer network. In some instances, the computer network comprises one or more computers operably connected to the digital optical device, wherein operably connected may be wireless or physical. In some implementations, the computer network comprises a plurality of computers and/or devices which are connected by physical or wireless means. A computer of the network may be located remotely from the digital optical device. In some instances, the computer network comprises one or more acquisition computers for controlling the acquisition of an image of the specimen. In exemplary embodiments, the computer network is configured to control the acquisition, processing and/or display of an image of the sample, wherein the image may be saved and/or live. In some instances, the network comprises one or more displays for viewing an acquired image, either saved, live or both. In some embodiments, one or more of the displays is a component of a viewing terminal of the network. In some embodiments, a specimen is viewed remotely from the digital optical device at a remote terminal, such as a viewing terminal.

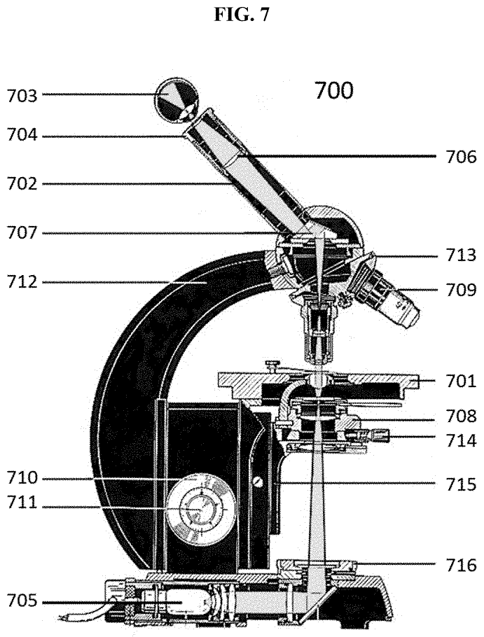

An exemplary digital optical device 700 useful in microscopy methods and systems described herein is shown in FIG. 7. Device 700 comprises a stage 701. The stage is configured to hold a specimen for viewing through tube 702 via an eye 703 and eyepiece 704. The specimen is illuminated for viewing using a halogen bulb 705 as a light source. Device 700 comprises the following optical components: lens 706, prism 707, and condenser 708. The specimen is viewed through one or more objectives 709, for example, a 4x objective. The view of the specimen is focused using a coarse focus 710 and a fine focus 711. Device 700 further comprises an arm 712, nosepiece 713, aperture diaphragm 714, condenser focus 715, and field diaphragm 716.

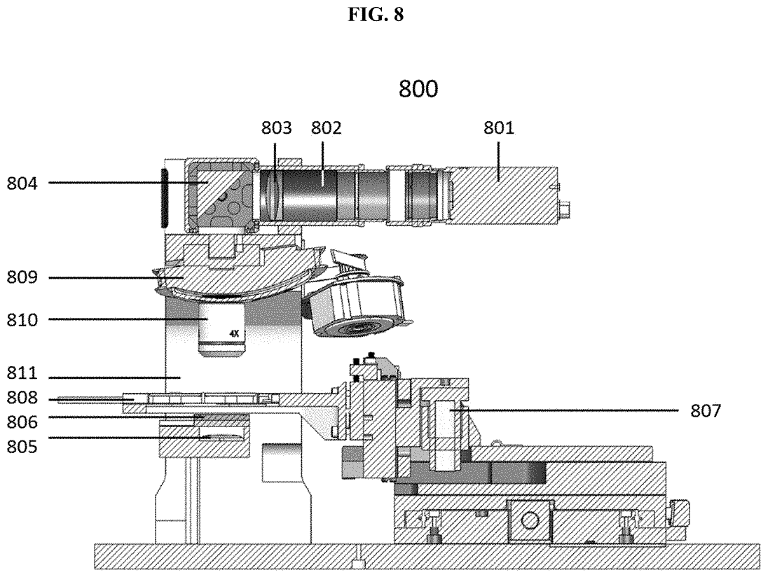

A detailed view of an exemplary digital optical device 800 comprising an LED system useful in microscopy methods and systems described herein is shown in FIG. 8. Device 800 is operably connected to an imaging device (e.g., camera) 801 at one end of a viewing tube 802. Device 800 comprises the following optical components: tube lens 803, prism 804, LED array 805, and diffuser 806. Device 800 comprises a focus motor 807 for focusing a view of a specimen presented on stage 808. Device 800 further comprises a nosepiece 809, objective 810, and arm 811.

### Instructions

In various aspects, a device described herein is controlled by a user submitting an instruction to a control computer operably connected to the device. In some embodiments, the control computer is a remote computer at a location different from the device, wherein the device and the remote computer are operably connected via a computer network. In some cases, a user instruction is submitted to a control computer to move a stage of a device. For example, to position the stage and/or to focus a view of a specimen on the stage. In some embodiments, an instruction is submitted to a control computer to acquire a micrograph of a view of a specimen using an imaging device, wherein the imaging device is a component of, or operably connected to, an optical digital device. In some embodiments, an instruction is submitted to a control computer to position a slide of a device relative to an image map created by a preview collection. In some embodiments, an instruction is submitted to a control computer to focus a view of a specimen through a digital optical device. For example, instructions to focus up and/or focus down. In some embodiments, an instruction is submitted to a control computer to take and save an image of a specimen using an imaging device and a digital optical device. In some embodiments, an instruction is submitted to a control computer to define an area of a specimen for viewing through a digital optical device at a predetermined or settable speed. In some embodiments, an instruction is submitted to a control computer to control movement of a digital optical device so that an area of a specimen for viewing is displayed in frames at a fixed or defined interval. In some embodiments, an instruction is submitted to a control computer to change a magnification of a digital optical device. In some embodiments, an instruction is submitted to a control computer to adjust image settings of a digital optical device. In some embodiments, an instruction is submitted to a control computer to automatically focus a view of a specimen through a digital optical device. In some embodiments, an instruction is submitted to a control computer to automatically focus a view of a specimen through a digital optical device at one or more focal points, record the focal points, and apply the focal points to a surface map to correlate with an X/Y position of the specimen. In some embodiments, an instruction is submitted to a control computer to eject a slide from a slide holder of a digital optical device. In some embodiments, an instruction is submitted to a control computer to send a message to a user to communicate that a procedure comprising viewing a specimen on a digital optical device is complete. In some cases, the message indicates that the digital optical device is ready to receive a next specimen. In some cases, the message is a text message or SMS message. In some cases, the message is an alarm.

### Specimens

A specimen includes, without limitation, biological samples which are traditionally viewed using microscopy in fields such as pathology, surgical pathology, surgery, veterinary medicine, education, life science research, anatomic pathology, cytology and cytopathology. In some embodiments, a specimen is a tissue sample. The specimens may be whole, cross-sections or any portion of a whole specimen. Specimens include samples which are not usually processed for traditional microscopy viewing on slides. Examples of such specimens include, without limitation, geological samples such as rocks of various sizes, metal based samples, and samples, e.g., opaque samples, which require differential illumination over traditional microscopy where light cannot be delivered through the specimen.

### Live Viewing History

In some aspects, devices and methods described herein document a user viewing a specimen through a digital optical device. In some embodiments, this documentation comprises a history of every change that occurs in the device while the user is viewing the specimen. This includes, without limitation, the positional state of the instrument, which includes x, y, and Z locations, magnification, and timestamp. This can be loaded later and the session can be recreated. In some embodiments, the recall of these steps do not rely on taking pictures or a video, but a video can be produced at a later time by loading the history file and recording the frames created of a previously recorded session. The data may also be based on feedback from encoders, as well as from a poll of the system state of the exact positions, magnification, and time every time a change is made. The history may also be taken both locally and remotely. For instance, if a user, for example a medical resident, is having trouble interpreting or reading a slide, the user can forward the slide and session to a consultant, such as a consulting physician, who now, for the first time, not only knows what the slide says, but the exact steps the user (e.g., medical resident) took to view the slide and how to advise the user where the decision making was flawed.

### Video and Audio Files

Many video formats are suitable including, by way of non-limiting examples, Windows Media Video (WMV), Windows® Media®, Motion Picture Experts Group (MPEG), Audio Video Interleave (AVI), Apple® QuickTime®, RealMedia®, Flash Video, Motion JPEG (M-JPEG), WebM, and Advanced Video Coding High Definition (AVCHD). In some embodiments, video is uncompressed (e.g., RAW format). In other embodiments, video is compressed. Both lossy and lossless video CODECs are suitable including, by way of non-limiting examples, DivX™, Cineform, Cinepak, Dirac, DV, FFV], H.263, H.264, H.264 lossless, JPEG 2000, MPEG-I, MPEG-2, MPEG4, On2 Technologies (VPS, VP6, VP7, and VP8), RealVideo, Snow lossless, Sorenson Video, Theora, and Windows Media Video (WMV).

In some embodiments, suitable video media is standard-definition. In further embodiments, a standard-definition video frame includes about 640×about 480 pixels, about 640×about 380, about 480×about 320 pixels, about 480×about 270 pixels, about 320×about 240 pixels, or about 320×about 180 pixels. In other embodiments, suitable video media is high-definition. In further embodiments, a high-definition video frame includes at least about 1280×about 720 pixels or at least about 1920×about 1080 pixels.

Many audio formats are suitable including, by way of non-limiting examples, MP3, WAV, AIFF, AU, Apple R Lossless, MPEG-4, Windows Media®, Vorbis, AAC, and Real Audio.

### Presentations

In some embodiments, the methods, systems and devices described herein generate an automatic presentation comprising acquired images of a specimen. A presentation includes any media that can display an acquired image with appropriate text. In some embodiments, a presentation automatically generated herein is a file configured for use with a presentation viewer such as PowerPoint, Sway, or Google Slides. In some embodiments, a presentation automatically generated herein is editable in a presentation viewer.

In some embodiments, a presentation may be created automatically as an output from the device, which includes all preview images automatically placed in position. Those preview images, for example, are automatically hyperlinked to another part of the document which includes a thumbnail of each image taken from the slide, with the corresponding X/Y position where the image was taken noted on an enlarged image of the preview slide. Each thumbnail may be linked to the full image taken and text notes which were taken during the acquisition process are automatically embedded into each image. This allows for a user or practitioner to take images at will while using the device, and automatically assemble all images and relevant instrument data into a format which can be presented to others for consultation or discussion and presentation.

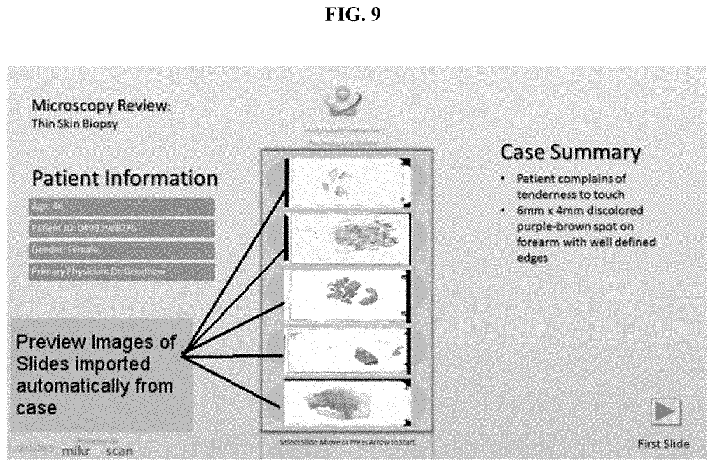

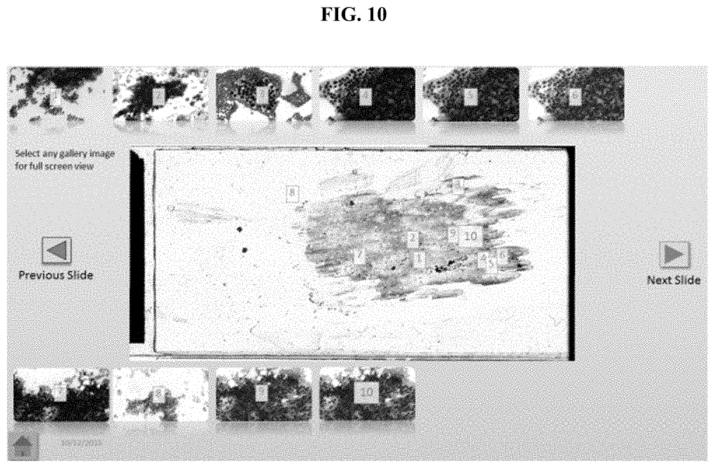

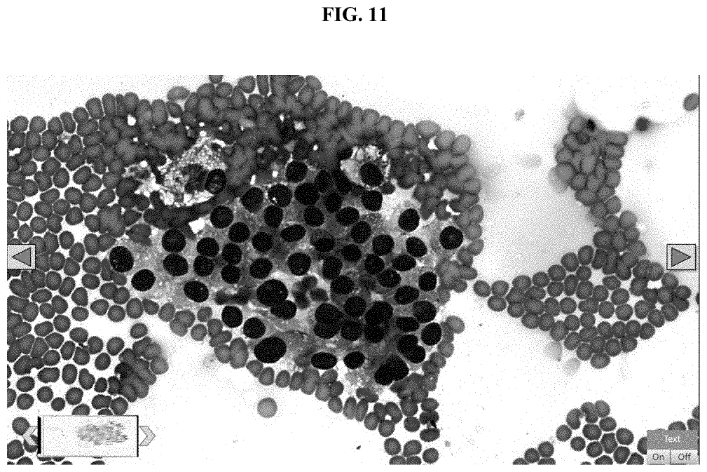

An exemplary presentation is shown in the slides of FIGS. 9-11. FIG. 9 shows a screenshot of a presentation comprising images of a clinical specimen acquired using a digital optical device described herein. The images are generated by a user instructing a control computer to acquire preview images of the specimen with the device. The presentation software presents with the preview images data corresponding to the images. As shown in FIG. 9, the corresponding data comprises patient information and a case summary. FIG. 10 shows a screenshot of a presentation comprising a low resolution image of a specimen having annotations, wherein 10 distinct regions of the specimen have been imaged at a high magnification. FIG. 11 shows a high magnification image of a specimen acquired using a digital optical device that has been uploaded automatically into a presentation slide. Annotations describing the specimen made by a user during viewing are uploaded automatically with the image.

### Digital Processing Device

In some embodiments, the methods, systems, media, and devices described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the nonvolatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Non-Transitory Computer Readable Storage Medium

In some embodiments, the methods, systems, media, and devices disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the methods, systems, media, and devices disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™ and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windowsx Marketplace for Mobile, Ovi Store for Nokiax devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the methods, systems, media, and devices disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the methods, systems, media, and devices disclosed herein include one or more databases, or use of the same. In view of the disclosure provided herein, those of skill in the art will recognize that many databases are suitable for storage and retrieval of specimen, user, location, positioning, focus, magnification, and presentation information. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### EXAMPLES

The following illustrative examples are representative of embodiments of the software applications, systems, and methods described herein and are not meant to be limiting in any way.

### Example 1—Focusing Using a Digital Optical Device

A digital optical device is used to focus a view of a specimen. The specimen is placed on the slide mount of the optical device 100 shown in FIG. 1. The specimen is viewed by a user at the device and the device is controlled by the user with a control computer. The device comprises one or more optical components including a low and high objective lens. The user views the specimen using a low objective lens. The user controls the view of the specimen by instructing the control computer to move the slide mount in an X-axis and Y-axis until an area of interest of the specimen is identified. A focused view of the area of interest is achieved by the user instructing the control computer to move slide mount 102 in a Z-axis between positions 103 and 104 at a low objective, while the remaining components of the device remain stationary (i.e. no optical components are moved in a Z-axis). To generate a finer focused view of the specimen, the user optionally instructs the device, via the control computer, to change the objective lens to a higher power lens and control the movement of the slide mount in a Z-axis until the user views a focused image of the area of interest. Again, the remaining components of the device remain stationary. The user instructs an imaging device connected to the optical device and controlled by the control computer, to acquire an image of the focused view of the area of interest.

### Example 2—Remote Focusing Using a Digital Optical Device

A digital optical device is used to focus a view of a specimen as described in Example 1 and FIG. 1. The specimen is viewed by a user at a location remote from the device and the device is controlled by the user with a remote computer. The remote user views the specimen using a low objective lens. The user remotely controls the view of the specimen by moving the slide mount in an X-axis and Y-axis until an area of interest of the specimen is identified. A focused view of the area of interest is achieved by the remote user sending a command to the device via the remote computer to move slide mount 102 in a Z-axis between positions 103 and 104 at a low objective, while the remaining components of the device remain stationary (i.e. no optical components are moved in a Zaxis). To generate a finer focused view of the specimen, the user optionally instructs the device, via the remote computer, to change the objective lens to a higher power lens and remotely controls the movement of the slide mount in a Z-axis until the user views a focused image of the area of interest. Again, the remaining components of the device remain stationary. The user instructs an imaging device connected to the optical device and controlled remotely by the remote computer, to acquire an image of the focused view of the area of interest.

### Example 3—Documentation of an Image by Digital Microscopy

A specimen having an area of interest with multiple depths is viewed using a digital optical device as shown in FIG. 1. The specimen is placed on the slide mount of the device and a user remote from the device location views a digital image of the specimen in real time. The remote user controls the device using a remote computer. The remote user moves the slide mount in X- and Y-axes until the area of interest is identified. The user controls movement of the slide mount in a Z-axis to identify the top and bottom focal planes of the area of interest. The user instructs an imaging device coupled to the optical device to acquire a given number of images of the area of interest at different depths between the top and bottom focal planes. The images are stored on a computer readable media. A second user uploads the stored images on a computer comprising software that displays a view of the images. The second user commands the computer to display the images so that the second user can focus through the images at varying depths as if the second user were viewing the area of interest in real time.

### Example 4—Microscopy Recordation

A first user remotely views a specimen using a digital optical device and records the viewing session onto a video file. A second user views the video file and optionally repeats the viewing process of the first user. The first user views the specimen positioned on a slide stage of a digital optical device on a remote viewing station comprising a remote viewer (e.g., computer screen) and a remote computer. The remote viewing station is connected to the digital optical device via a computer network. The first user views the specimen in real time by instructing the device through the remote computer to move the specimen so that different areas of interest of the specimen are viewable. Focused views of the specimen are obtained by the first user instructing the device to move the slide stage in a Z-axis. The user instructs a computer to record micrographs of the specimen and data corresponding to each micrograph, including, X, Y and Z positions, time and magnification in a file history. The file history is saved with specimen details in a case file on a server of the computer network. A second user opens the case file on a second user computer and views a video of the recorded micrographs.

### Example 5—Specimen Evaluation Using Remote Digital Microscopy

A user views a specimen by advancing a field of view of a digital optical device in a defined pattern so that the user views each region of a define area of the specimen. The specimen is presented on a slide to a stage of the digital optical device. The digital optical device is connected to a remote computer controllable by the user with a remote computer. The user instructs the device, via the remote computer, to advance the stage in a pattern shown in FIG. 3. The device moves the stage in the X- and Y-axes over a defined period of time so that the user views all regions of the defined area of the specimen.

### Example 6—LED Illumination System

The microscopy methods described in Examples 1-5 are performed using a digital optical device comprising an LED illumination system. The LED illumination system of the microscope is shown in FIG. 5 and comprises an LED as a light source and a holographic light shaping diffuser.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

What is claimed is:

1. A computer-implemented method of focusing digital optical devices, the method comprising:
(a) transmitting, by a computer at a first location, a focusing instruction to a digital optical device at a second location, the focusing instruction comprising one or more commands for the digital optical device to move a slide and a slide mount in a Z-axis to focus a digital optical image; and
(b) receiving, by the computer, a focused digital optical image from the digital optical device; provided that the digital optical device moves only the slide and the slide mount in the Z-axis in response to the focusing instruction.

2. The method of claim 1, wherein the second location is different from the first location.

3. The method of claim 1, wherein the focusing instruction is sent via a computer network.

4. The method of claim 1, wherein the digital optical device comprises a telemicroscope.

5. A computer-implemented method of documenting specimen of interest imaged by a digital optical device comprising:
(a) transmitting, by a computer at a first location, a first focusing instruction to a digital optical device at a second location, the focusing instruction comprising a command for the digital optical device to focus on a top-most plane of an image having a plurality of focus planes;
(b) transmitting, by the computer, a second focusing instruction to the digital optical device, the focusing instruction comprising a command for the digital optical device to focus on a predetermined number of steps above and below the focus point of the image;
(c) determining, by the computer, a depth of field of the image and an optimal step size based on the depth of field;
(d) receiving, by the computer, a sequential series of images, each image created at a different focus plane separated from adjacent planes by the step size;
(e) presenting, by the computer, an interface to allow a user at the first location to identify a specimen of interest within the sequential series of images and define a depth of the specimen; and
(f) generating, by the computer, a document comprising a plurality of the sequential series of images spanning the depth of the specimen, each image on a separate page of the document.

6. The method of claim 5, wherein the second location is different from the first location.

7. The method of claim 5, wherein the focusing instructions are sent via a computer network.

8. The method of claim 5, wherein the digital optical device is a telemicroscope.

9. A computer-implemented method of recording a live viewing history of a specimen evaluated at a digital optical device comprising:
(a) receiving, by a computer at a first location, one or more micrographs of the specimen, the one or more micrographs generated by a digital optical device at a second location;
(b) receiving, by the computer, a plurality of data describing a live viewing session of the specimen at the digital optical device, the plurality of data comprising X- and Y position of stage, focus, and magnification of the digital optical device captured and the time interval taken at each step;
(c) generating, by the computer, a live viewing history from the plurality of data; and
(d) applying, by the computer, the live viewing history to the one or more micrographs of the specimen to output a video file that replicates the live viewing session.

10. The method of claim 9, wherein the second location is different from the first location.

11. The method of claim 9, wherein the time interval is user-defined.

12. The method of claim 9, further comprising presenting, by the computer, an interface allowing a user at the first location to record voice audio and wherein the outputting of the video file comprises integrating the voice audio into the video file.

13. The method of claim 9, further comprising comparing, by the computer, a total tissue of the specimen to tissue viewed in the live viewing session to generate a score.

14. The method of claim 9, further comprising: creating a vector trail of the X- and Y-position of stage and focus of the digital optical device relative to the specimen; and overlaying the vector trail on the one or more micrographs of the specimen.

15. The method of claim 9, further comprising viewing the one or more micrographs as a live stream of constantly refreshing images.

16. A computer-implemented method of evaluating a specimen at a digital optical device comprising:
(a) receiving, by a computer at a first location, one or more micrographs of the specimen, the one or more micrographs generated by a digital optical device at a second location;
(b) presenting, by the computer, an interface allowing a user at the first location to define a total viewing area for the specimen;
(c) separating, by the computer, the total viewing area into a plurality of fields of view; and
(d) transmitting, by the computer, instructions to the digital optical device, the instructions comprising one or more commands for the digital optical device to move a stage of the device to advance through the fields of view at a repeating time interval.

17. The method of claim 16, wherein the second location is different from the first location.

18. The method of claim 16, wherein the time interval is user-defined.

19. The method of claim 16, wherein the instructions comprise one or more commands for the digital optical device to move the stage to advance through the fields of view in a pattern corresponding to rows across the total viewing area.

20. The method of claim 16, wherein the instructions comprise one or more commands for the digital optical device to move the stage to advance through the fields of view in a pattern corresponding to columns across the total viewing area.

21. The method of claim 16, wherein the method further comprises automatically determining the area of a total of tissue detected in the specimen.

22. A computer-implemented method of automatically generating a presentation or a report on the evaluation of a specimen at a digital optical device comprising:
(a) receiving, by a computer at a first location, a color preview micrograph of the specimen, the preview micrograph generated by a digital optical device at a second location;
(b) performing, by the computer, a white balance on the preview micrograph;
(c) determining, by the computer, the dominant colors in the preview micrograph;
(d) defining, by the computer, an area to scan based on detection of a contiguous region of the preview micrograph including the dominant colors; e. generating, by the computer, a plurality of focus points within the area to scan; and
(e) evaluating, by the computer, each focus point to determine if it is above tissue of the specimen based on the dominant colors; and g. adjusting, by the computer, the position of any focus points that are not over tissue of the specimen and repeating the evaluation.

23. The method of claim 22, wherein the second location is different from the first location.

24. The method of claim 22, wherein the determining of the dominant colors in the preview micrograph comprises determining a modal value of the colors and subsequently applying a white threshold, a black threshold, a color threshold, and a paleness threshold.

25. A computer-implemented method of automatically generating a presentation or a report on the evaluation of a specimen at a digital optical device comprising:
(a) storing, by a computer at a first location, one or more presentation templates;
(b) receiving, by the computer, a color preview micrograph of the specimen, the preview micrograph generated by a digital optical device at a second location;
(c) receiving, by the computer, one or more high-magnification micrographs of the specimen, the one or more high-magnification micrographs optionally associated with text annotation; and
(d) generating, by the computer, the presentation by integrating the color preview micrograph of the specimen and the one or more high-magnification micrographs into a selected presentation template, the presentation comprising the color preview micrograph, the color preview micrograph linked to the one or more high magnification micrographs and the associated text annotations, if any, the links indicating the position within the specimen each high-magnification micrograph was created.

26. The method of claim 25, wherein the second location is different from the first location.

27. The method of claim 25, wherein the method further comprises presenting an interface allowing a user at the first location to integrate one or more previously generated presentations into the presentation.

28. A computer-implemented method of illuminating a specimen within a digital optical device comprising positioning an LED array on a side of the specimen opposite an imaging mechanism of the digital optical device, and placing a holographic light diffusing substrate between the LED array and the specimen.

29. A digital optical device comprising:
(a) an electromagnet;
(b) a stage; and
(c) a specimen eject mechanism;
wherein the electromagnet is configured to fix a position of the stage when the specimen eject mechanism is activated.

30. The digital optical device of claim 29, wherein the digital optical device comprises a microscope.

31. The digital optical device of claim 29, wherein the microscope comprises a remotely operated telemicroscope.

32. The digital optical device of claim 29, further comprises a whole slide imaging scanner.

33. A digital optical device comprising:
(a) a memory;
(b) an optical array;
(c) a stage;
(d) a digital image capture unit; and
(e) a motorized positioning unit;
wherein X-, Y-, and Z-positions of the optical array relative to the stage are stored in the memory upon each activation of the digital image capture unit, and the motorized positioning unit is configured to return the optical array to recorded positions associated with a particular digital image upon a request from a user.

34. The digital optical device of claim 33, wherein the digital optical device comprises a microscope.

35. The digital optical device of claim 33, wherein the microscope comprises a remotely operated telemicroscope.

36. The digital optical device of claim 33, wherein the Y-position further comprises a report on a slide among a plurality of slides being viewed.

37. A computer-implemented system for telemicroscopy comprising:
(a) a digital optical device comprising a slide mount having a range of Z-axis focus between a first position and a second position; a motor for moving the slide mount within the Z-axis focus range; a light source; and an optical component;
(b) a digital processing device comprising at least one processor, an operating system configured to perform executable instructions, a memory, and a computer program including instructions executable by the digital processing device to create a telemicroscopy focus application comprising:
(1) a software module instructing the motor of the digital optical device to move in a Z-axis in a number of steps between the first position and the second position to focus a digital optical image, wherein the digital optical image is focusable; and
(2) a software module receiving the digital optical image from the digital optical image device;
wherein the digital optical device is located at a first location and the digital processing device is located at a second location; and wherein the digital processing device and the digital optical device send and receive instructions, respectively, over a communication network.

38. The system of claim 37, wherein the second location is different from the first location.

39. The system of claim 37, wherein the digital optical device comprises an imaging device and wherein the application comprises a software module instructing the imaging device to acquire a micrograph of the focused digital optical image.

40. The system of claim 37, wherein the application comprises a software module instructing the digital optical device to import the acquired micrograph into a presentation or a report.

41. The system of claim 37, wherein the light source comprises a LED light and the optical component comprises a light shaping diffuser.

42. Non-transitory computer-readable storage media encoded with a computer program including instructions executable by a processor to create an application for focusing a digital optical device, the application comprising a software module instructing a motor of the digital optical device to move in a Z-axis in a fixed number of steps between a first position and a second position to create a focusable digital optical image; and a software module receiving a focused digital optical image from the digital optical image device; wherein the digital optical device is located at a first location and the digital processing device is located at a second location; and wherein the digital processing device and digital optical device send and receive instructions, respectively, over a telecommunication network.

43. The storage media of claim 42, wherein the second location is different from the first location.

44. The storage media of claim 42, wherein the application further comprise a software module instructing an imaging device operably connected to the digital optical device to acquire a micrograph of the focusable digital optical image.

45. A computer-implemented system for telemicroscopy comprising:
(a) a digital optical device comprising a slide mount having a range of Z focus between a first position and a second position; a motor for moving the slide mount within the Z focus range; a light source; and an optical component;
(b) a digital processing device comprising at least one processor, an operating system configured to perform executable instructions, a memory, and a computer program including instructions executable by the digital processing device to create a telemicroscopy focus application comprising:
(1) a software module instructing the motor of the digital optical device to move in a Z-axis between the first position and the second position to focus on the top-most plane of an image having a plurality of focus planes;
(2) a software module instructing the motor of the digital optical device to move in a Z-axis between the first position and the second position to focus on the bottommost plane of the image;
(3) a software module determining a depth of field of the image and an optimal step size based on the depth of field;
(4) software module receiving a sequential series of images, each image created at a different focus plane separated from adjacent planes by the step size;
(5) a software module presenting an interface to allow a user to identify a specimen of interest within the sequential series of images and define a depth of the specimen; and
(6) a software module generating a document comprising a plurality of the sequential series of images spanning the depth of the specimen, each image on a separate page of the document; wherein the digital optical device is located at a first location and the digital processing device and user are located at a second location; and wherein the digital processing device and digital optical device send and receive instructions, respectively, over a communication network.

46. The system of claim 45, wherein the second location is different from the first location.

47. The system of claim 45, wherein the light source comprises a LED light and the optical component comprises a light shaping diffuser.

48. Non-transitory computer-readable storage media encoded with a computer program including instructions executable by a processor to create an application for documenting a series of images with a digital optical device, the application comprising
(a) a software module instructing a motor of the digital optical device to move in a Z-axis between a first position and a second position to focus on the top-most plane of an image having a plurality of focus planes;
(b) a software module instructing the motor of the digital optical device to move in a Z-axis between the first position and the second position to focus on the bottom-most plane of the image;
(c) a software module determining a depth of field of the image and an optimal step size based on the depth of field; a software module receiving a sequential series of images, each image created at a different focus plane separated from adjacent planes by the step size;
(d) a software module presenting an interface to allow a user to identify a specimen of interest within the sequential series of images and define a depth of the specimen;
and a software module generating a document comprising a plurality of the sequential series of images spanning the depth of the specimen, each image on a separate page of the document;
wherein the digital optical device is located at a first location and the digital processing device and user are located at a second location; and wherein the digital processing device and digital optical device send and receive instructions, respectively, over a communication network.

49. The storage media of claim 48, wherein the second location is different from the first location.

50. The storage media of claim 48, wherein the application further comprises a software module instructing the digital optical device to import one or more of the series of images into a presentation or a report.

51. A computer-implemented system for telemicroscopy comprising:
(a) a digital optical device comprising a slide mount having a range of positions along a X- and Y-axis; a motor for moving the slide mount along the X- and Y-axis; a light source; and an optical component; and
(b) a digital processing device comprising at least one processor, an operating system configured to perform executable instructions, a memory, and a computer program including instructions executable by the digital processing device to create an application for recording a telemicroscopy viewing history comprising:
1) a software module receiving one or more micrographs of a specimen positioned on the slide mount of the digital optical device;
2) a software module receiving a plurality of data describing a live viewing session of the specimen, the plurality of data comprising X- and Y-position of the slide mount, focus, and magnification of the digital optical device captured repetitively at a time interval or at a time when a changed event occurs;
3) a software module generating a live viewing history from the plurality of data; and
4) a software module applying the live viewing history to the one or more micrographs of the specimen to output a video file that replicates the live viewing session;
wherein the digital optical device is located at a first location and the digital processing device is located at a second location; and wherein the digital optical device and digital processing device send and receive micrographs and data, respectively, over a telecommunication network.

52. The system of claim 51, wherein the second location is different from the first location.

53. The system of claim 51, wherein the light source comprises a LED light and the optical component comprises a light shaping diffuser.

54. The system of claim 51, wherein the application further comprises a software module instructing the digital optical device to import the video file into a presentation or a report.

55. The system of claim 51, wherein the time interval is user-defined.

56. The system of claim 51, wherein the time interval matches to a viewing history of an original user.

57. The system of claim 51, the application further comprises a software module presenting an interface allowing a user at the second location to record voice audio and wherein the outputting of the video file comprises integrating the voice audio into the video file.

58. The system of claim 51, wherein the application further comprises a software module comparing a total tissue of the specimen to tissue viewed in the live viewing session to generate a score.

59. The system of claim 51, wherein the application further comprises creating a vector trail of the X- and Y-positions of slide mount and focus of the digital optical device relative to the specimen; and overlaying the vector trail on the one or more micrographs of the specimen.

60. Non-transitory computer-readable storage media encoded with a computer program including instructions executable by a processor to create an application for recording a live viewing history of a specimen evaluated with a digital optical device, the application comprising
(a) a software module receiving one or more micrographs of a specimen positioned on the slide mount of the digital optical device;
(b) a software module receiving a plurality of data describing a live viewing session of the specimen, the plurality of data comprising X- and Y-position of the slide mount, focus, and magnification of the digital optical device captured repetitively and a timestamp;
(c) a software module generating a live viewing history from the plurality of data; and
(d) a software module applying the live viewing history to the one or more micrographs of the specimen to output a video file that replicates the live viewing session; wherein the digital optical device is located at a first location and the digital processing device is located at a second location; and wherein the digital optical device and digital processing device send and receive micrographs and data, respectively, over a communication network.

61. The storage media of claim 60, wherein the second location is different from the first location.

62. The storage media of claim 60, wherein the application further comprises a software module instructing the digital optical device to import the video file into a presentation or a report.

63. The storage media of claim 60, wherein the time interval is user-defined.

64. The storage media of claim 60, the application further comprises a software module presenting an interface allowing a user at the second location to record voice audio and wherein the outputting of the video file comprises integrating the voice audio into the video file.

65. The storage media of claim 60, wherein the application further comprises a software module comparing a total tissue of the specimen to tissue viewed in the live viewing session to generate a score.

66. The storage media of claim 60, wherein the application further comprises creating a vector trail of the X- and Y-positions of slide mount and focus of the digital optical device relative to the specimen; and overlaying the vector trail on the one or more micrographs of the specimen.

67. A computer-implemented system for telemicroscopy comprising:
(a) a digital optical device comprising a slide mount having a range of positions along a X- and Y-axis defining fields of view of a specimen positioned on the slide mount; a motor for moving the slide mount along the X- and Y-axis; a light source; and an optical component; and
(b) a digital processing device comprising at least one processor, an operating system configured to perform executable instructions, a memory, and a computer program including instructions executable by the digital processing device to create a specimen evaluation application comprising:
1) a software module receiving one or more micrographs of the specimen, the one or more micrographs generated by the digital optical device;
2) a software module presenting an interface allowing a user to define a total viewing area for the specimen;
3) a software module separating the total viewing area into a plurality of fields of view; and
4) a software module instructing the motor to move the slide mount to advance through the fields of view at a repeating time interval;
wherein the digital optical device is located at a first location and the digital processing device and user are located at a second location; and wherein the digital optical device and digital processing device send and receive information over a telecommunication network.

68. The system of claim 67, wherein the second location is different from the first location.

69. The system of claim 67, wherein the light source comprises a LED light and the optical component comprise a light shaping diffuser.

70. The system of claim 67, wherein the time interval is user-defined.

71. The system of claim 67, wherein the instructions comprise one or more commands for the motor to move the slide mount to advance through the fields of view in a pattern corresponding to rows across the total viewing area.

72. The system of claim 67, wherein the instructions comprise one or more commands for the motor to move the slide mount to advance through the fields of view in a pattern corresponding to columns across the total viewing area.

73. The system of claim 67, wherein the application further comprises a software module automatically determining the area of a total of tissue detected in the specimen.

74. The system of claim 67, wherein the instructions comprise one or more commands for the motor to move the slide mount to advance through the fields of view in a straight line between two user defined points

75. Non-transitory computer-readable storage media encoded with a computer program including instructions executable by a processor to create an application for evaluating a specimen with a digital optical device, the application comprising
(a) a software module receiving one or more micrographs of the specimen, the one or more micrographs generated by the digital optical device;
(b) a software module presenting an interface allowing a user to define a total viewing area for the specimen;
(c) a software module separating the total viewing area into a plurality of fields of view; and
(d) a software module instructing the motor to move the slide mount to advance through the fields of view at a repeating time interval;
wherein the digital optical device is located at a first location and the digital processing device and user are located at a second location; and wherein the digital optical device and digital processing device send and receive information over a telecommunication network.

76. The storage media of claim 75, wherein the second location is different from the first location.

77. The storage media of claim 75, wherein the light source comprises a LED light and the optical component comprises a light shaping diffuser.

78. The storage media of claim 75, wherein the time interval is user-defined.

79. The storage media of claim 75, wherein the instructions comprise one or more commands for the motor to move the slide mount to advance through the fields of view in a pattern corresponding to rows across the total viewing area.

80. The storage media of claim 75, wherein the instructions comprise one or more commands for the motor to move the slide mount to advance through the fields of view in a pattern corresponding to columns across the total viewing area.

81. The storage media of claim 75, wherein the application further comprises a software module automatically determining the area of a total of tissue detected in the specimen.

82. The storage media of claim 75, wherein the travel is a user-defined straight line, and the system adjusts its speed to display all frames from one point to the other during travel.

83. A computer-implemented system for telemicroscopy comprising:
(a) a digital optical device comprising a slide mount for holding a specimen and a scanning imaging device; and
(b) a digital processing device comprising at least one processor, an operating system configured to perform executable instructions, a memory, and a computer program including instructions executable by the digital processing device to create a presentation application comprising:
1) a software module receiving a color preview micrograph of the specimen, the preview micrograph generated by the digital optical device;
2) a software module performing a white balance on the preview micrograph;
3) a software module determining the dominant colors in the preview micrograph;
4) a software module defining an area to scan based on detection of a contiguous region of the preview micrograph including the dominant colors;
5) a software module generating a plurality of focus points within the area to scan;
6) a software module evaluating each focus point to determine if it is above tissue of the specimen based on the dominant colors; and
7) a software module adjusting the position of any focus points that are not over tissue of the specimen and repeating the evaluation;
wherein the digital optical device is located at a first location and the digital processing device is located at a second location; and wherein the digital processing device receives the preview micrograph over a telecommunication network.

84. The system of claim 83, wherein the second location is different from the first location.

85. The system of claim 83, wherein the determining of the dominant colors in the preview micrograph comprises determining a modal value of the colors and subsequently applying a white threshold, a black threshold, a color threshold, and a paleness threshold.

86. Non-transitory computer-readable storage media encoded with a computer program including instructions executable by a processor to create an application for automatically generating a presentation or a report on the evaluation of a specimen at a digital optical device, the application comprising:
(a) a software module receiving a color preview micrograph of the specimen, the preview micrograph generated by the digital optical device;
(b) a software module performing a white balance on the preview micrograph;
(c) a software module determining the dominant colors in the preview micrograph;
(d) a software module defining an area to scan based on detection of a contiguous region of the preview micrograph including the dominant colors;
(e) a software module generating a plurality of focus points within the area to scan;
a software module evaluating each focus point to determine if it is above tissue of the specimen based on the dominant colors; and
(g) a software module adjusting the position of any focus points that are not over tissue of the specimen and repeating the evaluation;
wherein the digital optical device is located at a first location and the digital processing device is located at a second location; and wherein the digital processing device receives the preview micrograph over a telecommunication network.

87. The storage media of claim 86, wherein the second location is different from the first location.

88. The storage media of claim 86, wherein the determining of the dominant colors in the preview micrograph comprises determining a modal value of the colors and subsequently applying a white threshold, a black threshold, a color threshold, and a paleness threshold.

89. A computer-implemented system for telemicroscopy comprising:
(a) a digital optical device comprising a slide mount for holding a specimen and a scanning imaging device;
(b) a digital processing device comprising at least one processor, an operating system configured to perform executable instructions, a memory, and a computer program including instructions executable by the digital processing device to create a presentation application comprising:
1) a software module storing one or more presentation templates;
2) a software module receiving a color preview micrograph of the specimen, the preview micrograph generated by the digital optical device;
3) a software module receiving one or more high-magnification micrographs of the specimen, the one or more high-magnification micrographs optionally associated with text annotation; and
4) a software module generating the presentation by integrating the color preview micrograph of the specimen and the one or more high-magnification micrographs into a selected presentation template, the presentation comprising the color preview micrograph, the color preview micrograph linked to the one or more high-magnification micrographs and the associated text annotations, if any, the links indicating the position within the specimen each high-magnification micrograph was created;
wherein the digital optical device is located at a first location and the digital processing device is located at a second location; and wherein the digital processing device receives the preview and high-magnification micrographs over a telecommunication network.

90. The system of claim 89, wherein the second location is different from the first location.

91. The system of claim 89, wherein the application further comprises a software module presenting an interface allowing a user at the first location to integrate one or more previously generated presentations into the presentation.

92. Non-transitory computer-readable storage media encoded with a computer program including instructions executable by a processor to create an application for automatically generating a presentation or a report on the evaluation of a specimen at a digital optical device, the application comprising:
(a) a database comprising one or more presentation templates;
(b) a software module storing one or more presentation templates;
(c) a software module receiving a color preview micrograph of the specimen, the preview micrograph generated by the digital optical device;
(d) a software module receiving one or more high-magnification micrographs of the specimen, the one or more high-magnification micrographs optionally associated with text annotation; and
(e) a software module generating the presentation by integrating the color preview micrograph of the specimen and the one or more high-magnification micrographs into a selected presentation template, the presentation comprising the color preview micrograph, the color preview micrograph linked to the one or more high-magnification micrographs and the associated text annotations, if any, the links indicating the position within the specimen each high-magnification micrograph was created;
wherein the digital optical device is located at a first location and the digital processing device is located at a second location; and wherein the digital processing device receives the preview and high-magnification micrographs over a telecommunication network.

93. The system of claim 92, wherein the second location is different from the first location.

94. The system of claim 92, wherein the application further comprises a software module presenting an interface allowing a user at the first location to integrate one or more previously generated presentations into the presentation.
